(19) United States
(12) Patent Application Publication
(10) Pub. No.: US 2012/0251927 A1
(43) Pub. Date: Oct. 4, 2012

(54) HOLOGRAM-RECORDING MEDIUM

(75) Inventors: Norikatsu Sasao, Tokyo (JP); Kazuki Matsumoto, Tokyo (JP); Rumiko Hayase, Tokyo (JP); Yoshiaki Kawamonzen, Tokyo (JP); Masahiro Kanamaru, Tokyo (JP); Masaya Terai, Tokyo (JP)

(73) Assignee: KABUSHIKI KAISHA TOSHIBA, Tokyo (JP)

(21) Appl. No.: 13/524,850

(22) Filed: Jun. 15, 2012

Related U.S. Application Data
(63) Continuation of application No. PCT/JP2009/006904, filed on Dec. 16, 2009.

(51) Int. Cl. C07D 209/86; C07D 209/88; G03F 7/027

(52) U.S. Cl. 430/2; 548/440; 548/441

(57) ABSTRACT

A hologram-recording medium containing: a recording layer containing a polymerizable monomer containing a structural framework represented by the following general formula (1).

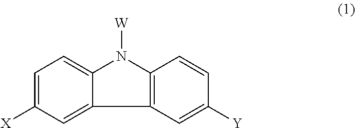

In general formula (1), X and Y are not the same, and X and Y are selected from hydrogen, iodine, bromine, and chlorine atoms and from methyl, ethyl, isopropyl, tert-butyl, phenyl, naphthyl, hydroxyl, methoxy, ethoxy, isopropoxy, tert-butoxy, phenoxy, naphthoxy, acetyl, carboxyl, acetoxy, thiophenyl, thionaphthyl, thiomethyl, thioethyl, thioisopropyl, thio-tert-butyl, and thiol groups; and W is selected from the group consisting of benzylvinyl, styryl, acryloyl, and methacryloyl groups.

CROSS-REFERENCE TO THE RELATED APPLICATION(S)

This is a Continuation Application of PCT Application No. PCT/JP2009/006904, filed on Dec. 16, 2009, which is published under PCT Article 21(2) in Japanese, the entire contents of which are incorporated herein by reference.

### FIELD

Embodiments described herein generally relates to a hologram-recording medium.

### BACKGROUND

It is known that a hologram-recording medium includes a recording layer which contains a three-dimensionally crosslinked polymeric matrix, a photo-radical generator, and a compound capable of ring-opening polymerization having an aliphatic cyclic structure that undergoes free-radical ring-opening polymerization, and it is also known that vinylcarbazole, which is a carbazole derivative, is used as a material for the recording medium.

### BRIEF DESCRIPTION OF THE DRAWINGS

A general configuration that implements the various features of the present invention will be described with reference to the drawings. The drawings and the associated descriptions are provided to illustrate embodiments of the invention and not to limit the scope of the invention.

FIG. 1 is a view which illustrates a hologram-recording medium according to a first embodiment of the invention.

FIG. 2 is a view which illustrates a hologram-recording medium according to a second embodiment.

FIG. 3 is a view which shows the configuration of an apparatus for hologram recording/reproducing.

FIG. 4 is a presentation showing the results of M/# evaluation of the hologram recording media according to Examples.

FIG. 5 is a presentation showing the results of M/# evaluation of the hologram recording media according to Examples.

FIG. 6 is a presentation showing the results of M/# evaluation of the hologram recording media according to Examples.

FIG. 7 is a presentation showing the results of M/# evaluation of the hologram recording media according to Examples.

FIG. 8 is a presentation showing the results of M/# evaluation of the hologram recording media according to Comparative Examples.

FIG. 9 is a presentation which shows a relationship between the concentration of a polymerizable monomer contained in the recording layer of a hologram-recording medium and M/#.

### DETAILED DESCRIPTION

According to the embodiments described herein, there is provided a hologram-recording medium according to one aspect of the invention which is characterized by including a recording layer which includes a polymerizable monomer containing a structural framework represented by the following general formula (1).

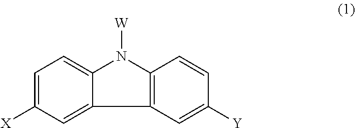

In general formula (1), X and Y are not the same, and X and Y are selected from hydrogen, iodine, bromine, and chlorine atoms and from methyl, ethyl, isopropyl, tert-butyl, phenyl, naphthyl, hydroxyl, methoxy, ethoxy, isopropoxy, tert-butoxy, phenoxy, naphthoxy, acetyl, carboxyl, acetoxy, thiophenyl, thionaphthyl, thiomethyl, thioethyl, thioisopropyl, thio-tert-butyl, and thiol groups; and W is selected from the group consisting of benzylvinyl, styryl, acryloyl, and methacryloyl groups.

Embodiments of the invention are explained below by reference to drawings. In the drawings explained below, like members or parts are designated by like numerals or signs, and duplicates of explanation are omitted.

### First and Second Embodiments

Hologram-recording media 10 according to first and second embodiments of the invention are explained.

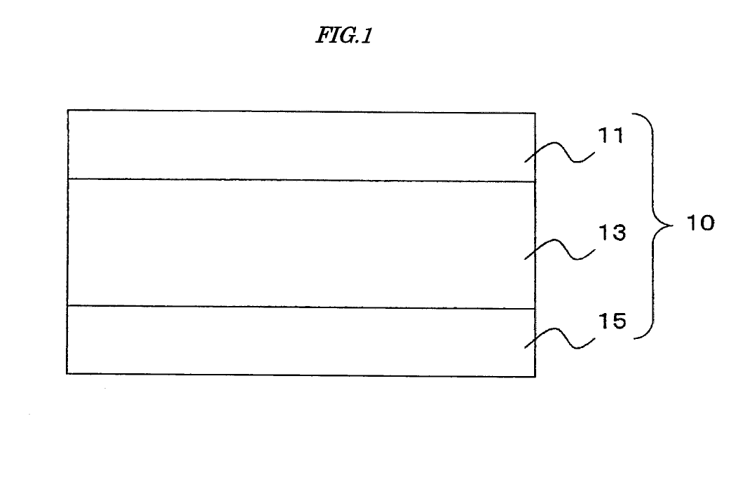

FIG. 1 is a view which illustrates a hologram-recording medium 10 according to this embodiment.

The hologram-recording medium 10 according to this embodiment has a structure in which a recording layer 13 for recording a hologram is held between two transparent substrates 11 and 15 which have light-transmitting properties.

The recording layer 13 includes a polymerizable monomer, a photopolymerization initiator, and a polymeric matrix. As the transparent substrates 11 and 15, they can be made of quartz, a glass, or a transparent resin.

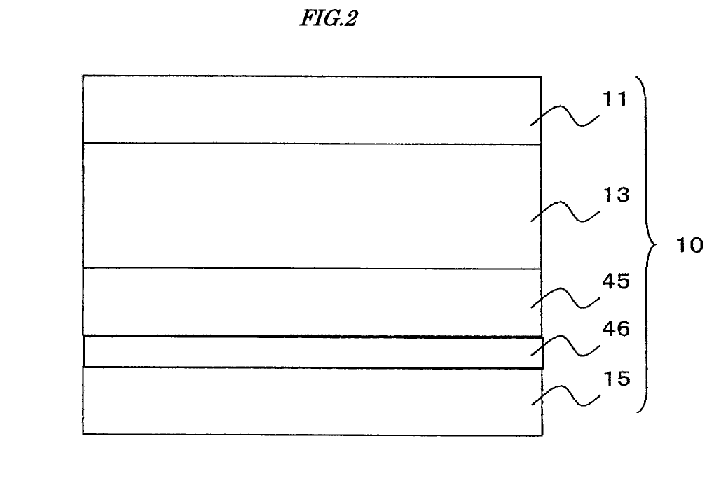

The hologram-recording medium 10 according to this embodiment can be a reflective hologram-recording medium which includes a reflecting layer 46 and a gap layer 45 that have been disposed between a transparent substrate 15 and a recording layer 13, as shown in FIG. 2. The reflecting layer 46 is constituted, for example, of aluminum, while a transparent resin or a glass, for example, is used for the gap layer 45. Thus, light can be converged at the recording layer 13.

In the hologram-recording media 10 according to these embodiments, information light and reference light are caused to interfere with each other within the recording layer 13 to thereby record a hologram. The hologram to be recorded in the hologram-recording medium 10 shown in FIG. 1 can be a transmission hologram recorded with information light and reference light which both strike on the recording layer through either of the transparent substrates 11 and 15. In the hologram-recording medium 10 shown in FIG. 2, a reflection hologram also can be formed by conducting recording in such a manner that incident light and information light are caused to strike through the transparent substrate 11 and reference light is reflected by the reflecting layer 46 toward the recording layer 13.

The interference between information light and reference light can be attained either two-beam interferometry or coaxial interferometry.

The materials of the recording layer 13 of the hologram-recording media 10 according to these embodiments are explained below.

The recording layer 13 includes one or more polymerizable monomers, a photopolymerization initiator, a polymeric matrix, etc. The polymerizable monomers may consist only of a polymerizable monomer represented by the following general formula (1) or may include both this polymerizable monomer and other polymerizable monomer(s).

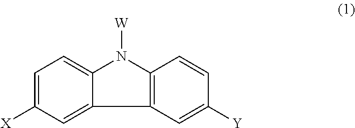

In general formula (1), X and Y are not the same, and X and Y are selected from hydrogen, iodine, bromine, and chlorine atoms and from methyl, ethyl, isopropyl, tert-butyl, phenyl, naphthyl, hydroxyl, methoxy, ethoxy, isopropoxy, tert-butoxy, phenoxy, naphthoxy, acetyl, carboxyl, acetoxy, thiophenyl, thionaphthyl, thiomethyl, thioethyl, thioisopropyl, thio-tert-butyl, and thiol groups; and W is selected from the group consisting of benzylvinyl, styryl, acryloyl, and methacryloyl groups.

The polymerizable monomer represented by general formula (1) has the framework of carbazole, and has different substituents respectively bonded in the 3-position and 6-position of the carbazole framework. The 3-position corresponds to the Y shown in general formula (1), and the 6-position corresponds to the X shown in general formula (1). Furthermore, the 9-position corresponds to the W shown in general formula (1).

Incidentally, the term “derivative” means a compound which has one or more substituents bonded to the framework.

The substituents bonded in the 3-position and the 6-position are not particularly limited, and are selected from hydrogen, iodine, bromine, and chlorine atoms and from methyl, ethyl, isopropyl, tert-butyl, phenyl, naphthyl, hydroxyl, methoxy, ethoxy, isopropoxy, tert-butoxy, phenoxy, naphthoxy, acetyl, carboxyl, acetoxy, thiophenyl, thionaphthyl, thiomethyl, thioethyl, thioisopropyl, thio-tert-butyl, and thiol groups. It is especially preferred that X and Y should be selected from hydrogen, iodine, bromine, and chlorine atoms and from phenyl, naphthyl, phenoxy, naphthoxy, thiophenyl, and thionaphthyl groups, among those substituents, because these substituents can improve the polarizability of the polymerizable monomer or increase the number of molecules per unit volume, resulting in an improvement in the refractive index of the polymerizable monomer. It is more preferred that X should be a hydrogen atom and Y should be selected from iodine, bromine, and chlorine atoms and from phenyl, naphthyl, phenoxy, naphthoxy, thiophenyl, and thionaphthyl groups. It can be seen that this combination is most preferred in view of the difference in size between the substituents. Specifically, the reason for the preference is that when one is a hydrogen atom, there is a considerable difference in physical dimension between this substituent and the other substituent, resulting in ease of the stacking which will be described later.

By thus bonding different substituents in the 3-position and the 6-position, the following effect is obtained. When layers of this polymerizable monomer are stacked so as to be oriented reversely, a relationship can be constructed in which the surface irregularities of the carbazole rings in one layer are mated with the surface irregularities of the carbazole rings in the other layer. As a result, layers of the polymerizable monomer can be stacked densely as compound with the case where the same substituent has been bonded in the 3-position and the 6-position, and this region can be made to have a further increased actual refractive index after recording, i.e., after polymerization reaction. As a result, a high degree of refractive-index modulation can be brought about through a smaller number of polymerization reaction operations. The smaller number of polymerization reaction operations means lower-energy irradiation. Namely, a hologram-recording medium having higher sensitivity can be provided according to the invention.

It is preferred that the polymerizable monomer represented by general formula (1) should be incorporated so that this monomer is contained in the recording layer in an amount of 0.1-50% by weight. In case where the amount thereof is less than 0.1% by weight, there is a possibility that a sufficient change in refractive index might not be obtained. In case where the amount thereof exceeds 50% by weight, there is a possibility that enhanced volumetric shrinkage might occur, resulting in a decrease in resolution. The content of the polymerizable monomer represented by general formula (1) is preferably 1-30% by weight, more preferably 1-15% by weight.

Examples of the polymerizable monomers other than the polymerizable monomer represented by general formula (1) include unsaturated carboxylic acids, unsaturated carboxylic acid esters, unsaturated carboxamides, and vinyl compounds.

Specific examples thereof include acrylic acid, methyl acrylate, ethyl acrylate, propyl acrylate, butyl acrylate, isobutyl acrylate, 2-ethylhexyl acrylate, octyl acrylate, lauryl acrylate, stearyl acrylate, cyclohexyl acrylate, bicyclopentenyl acrylate, phenyl acrylate, 2,4,6-tribromophenyl acrylate, isobornyl acrylate, adamantyl acrylate, methacrylic acid, methyl methacrylate, propyl methacrylate, butyl methacrylate, phenyl methacrylate, phenoxyethyl acrylate, chlorophenyl acrylate, adamantyl methacrylate, isobornyl methacrylate, N-methylacrylamide, N,N-dimethylacrylamide, N,N-methylenebisacrylamide, acryloylmorpholine, vinylpyridine, styrene, bromostyrene, chlorostyrene, tribromophenyl acrylate, trichlorophenyl acrylate, tribromophenyl methacrylate, trichlorophenyl methacrylate, vinyl benzoate, vinyl 3,5-dichlorobenzoate, vinylnaphthalene, vinyl naphthoate, naphthyl methacrylate, naphthyl acrylate, N-phenylmethacrylamide, N-phenylacrylamide, N-vinylpyrrolidinone, 1-vinylimidazole, bicyclopentenyl acrylate, 1,6-hexanediol diacrylate, pentaerythritol triacrylate, pentaerythritol tetraacrylate, dipentaerythritol hexaacrylate, diethylene glycol diacrylate, polyethylene glycol diacrylate, polyethylene glycol dimethacrylate, tripropylene glycol diacrylate, propylene glycol trimethacrylate, diallyl phthalate, and triallyl trimellitate.

Especially preferred polymerizable monomers other than the polymerizable monomer represented by general formula (1) are vinylnaphthalene, bromostyrene, chlorostyrene, tribromophenyl acrylate, trichlorophenyl acrylate, tribromophenyl methacrylate, and trichlorophenyl methacrylate, because these monomers are high in reactivity and refractive index. Suitable monomers may be selected from these while taking account of reactivity with the polymerizable monomer represented by general formula (1).

The photopolymerization initiator can be selected according to the wavelength of the recording light. Examples thereof include benzoin ethers, benzyl ketals, benzil, acetophenone derivatives, aminoacetophenone compounds, benzophenone derivatives, acylphosphine oxides, triazine compounds, imidazole derivatives, organic azide compounds, titanocene compounds, organic peroxides, and thioxanthone derivatives.

Specific examples thereof include benzil, benzoin, benzoin ethyl ether, benzoin isopropyl ether, benzoin butyl ether, benzoin isobutyl ether, 1-hydroxycyclohexyl phenyl ketone, benzyl methyl ketal, benzyl ethyl ketal, benzyl methoxyethyl ether, 2,2′-diethylacetophenone, 2,2′-dipropylacetophenone, 2-hydroxy-2-methylpropiophenone, p-tert-butyltrichloroacetophenone, thioxanthone, 1-chlorothioxanthone, 2-chlorothioxanthone, 2-methylthioxanthone, 2-isopropylthioxanthone, 3,3′,4,4′-tetra(t-butylperoxycarbonyl)benzophenone, 2,4,6-tris(trichloromethyl)-1,3,5-triazine, 2-(p-methoxyphenyl)-4,6-bis(trichloromethyl)-1,3,5-triazine, 2-[(p-methoxyphenyl)ethylene]-4,6-bis(trichloromethyl)-1,3,5-triazine, diphenyl(2,4,6-trimethylbenzoyl)phosphine oxide, 1-hydroxycyclohexyl phenyl ketone, 2-benzyl-2-(dimethylamino)-4′-morpholinobutyrophenone, 2,2-dimethoxy-1,2-diphenylethanone, bis(cyclopenta-1,3-dienyl)bis(1-(2,4-difluoro)-3H-pyrrol-3-yl)titanium, phenylbis(2,4,6-trimethylbenzoyl)phosphine oxide, 2-methyl-4′-methylthio-2-morpholinopropiophenone, mixtures of 2-hydroxy-2-methyl-1-phenyl-1-propanone and bis(2,6-dimethoxybenzoyl)(2,4,4-trimethylpentyl)phosphine oxide, mixtures of 1-hydroxycyclohexyl phenyl ketone and bis(2,6-dimethoxybenzoyl)(2,4,4-trimethylpentyl)phosphine oxide, di-t-butyl peroxide, dicumyl peroxide, t-butyl cumyl peroxide, t-butyl peroxyacetate, t-butyl peroxyphthalate, t-butyl peroxybenzoate, acetyl peroxide, isobutyryl peroxide, decanoyl peroxide, lauroyl peroxide, benzoyl peroxide, t-butyl hydroperoxide, cumene hydroperoxide, methyl ethyl ketone peroxide, and cyclohexanone peroxide.

In the case where a blue semiconductor laser is used as a light source of recording light, a titanocene compound such as bis(cyclopenta-1,3-dienyl)bis(1-(2,4-difluoro)-3H-pyrrol-3-yl)titanium is suitable as the photopolymerization initiator.

It is preferred that the photopolymerization initiator should be incorporated in such an amount that the hologram-recording medium has a recording-light transmittance of 10-95%. In case where the recording-light transmittance thereof is less than 10%, there is a possibility that this hologram-recording medium might be reduced in sensitivity and diffraction efficiency. In case where the recording-light transmittance thereof exceeds 95%, most of the recording light passes through the recording layer and there is hence a possibility that the information to be recorded cannot be sufficiently recorded. It is more preferred that the photopolymerization initiator should be incorporated in such an amount that the hologram-recording medium has a recording-light transmittance of 20-90%.

Furthermore, it is preferred that the photopolymerization initiator should be incorporated in an amount of 0.1-20% by weight based on the recording layer. In case where the amount thereof is less than 0.1% by weight, there is a possibility that a sufficient change in refractive index might not be obtained. On the other hand, in case where the amount thereof exceeds 20% by weight, there is a possibility that this recording layer might show too high light absorption, resulting in decreases in sensitivity and diffraction efficiency. It is more preferred that the amount of the photopolymerization initiator to be incorporated should be 0.1-10% by weight based on the recording layer.

The polymeric matrix described herein is the base material of the recording layer which not only provides a field where the polymerizable monomers, photopolymerization initiator, etc. react but also has the function of maintaining the volume of the recording layer. If the polymerizable monomers and the photopolymerization initiator are removed from the recording layer which has not undergone recording, the residual polymer can be considered to be a polymeric matrix. In the case where a polymerization inhibitor such as that which will be described later, a plasticizer, and a sensitizer are contained in the recording layer, the state which remains after these ingredients also have been removed can be considered to be a polymeric matrix.

A polymeric matrix formed by the following polymerization reaction can be used. Examples of the reaction include epoxy/amine polymerization, epoxy/acid anhydride polymerization, epoxy/mercaptane polymerization, polymerization of an unsaturated ester with an amine by Michael addition, urethane formation through isocyanate/hydroxyl reactions, urea formation through isocyanate/hydroxyl reactions, and ethynyl/azide combination. Especially preferred polymerization reactions are epoxy/amine polymerization and epoxy/acid anhydride polymerization from the standpoint that the reactions proceed mildly.

Examples of the epoxy include 1,4-butanediol diglycidyl ether, 1,5-pentanediol diglycidyl ether, 1,6-hexanediol diglycidyl ether, 1,7-heptanediol diglycidyl ether, 1,8-octanediol diglycidyl ether, 1,9-nonanediol diglycidyl ether, 1,10-decanediol diglycidyl ether, 1,11-undecanediol diglycidyl ether, 1,12-dodecanediol diglycidyl ether, diethylene glycol diglycidyl ether, polyethylene glycol diglycidyl ether, polypropylene glycol diglycidyl ether, neopentyl glycol diglycidyl ether, diepoxyoctane, resorcinol diglycidyl ether, bisphenol A diglycidyl ether, bisphenol F diglycidyl ether, 3,4-epoxycyclohexenylmethyl 3′,4′-epoxycyclohexenecarboxylate, and polydimethylsiloxanes terminated by epoxypropoxypropyl.

Examples of the compounds which react with the epoxy include ethylenediamine, diethylenetriamine, triethylenetetramine, tetraethylenepentamine, pentaethylenehexamine, hexamethylenediamine, menthenediamine, isophoronediamine, bis(4-amino-3-methyldicyclohexyl)methane, bis(aminomethyl)cyclohexane, N-aminoethylpiperazine, m-xylylenediamine, 1,3-diaminopropane, 1,4-diaminobutane, trimethylhexamethylenediamine, iminobispropylamine, bis(hexamethylene)triamine, 1,3,6-trisaminomehylhexane, dimethylaminopropylamine, aminoethylethanolamine, tri(methylamino)hexane, m-phenylenediamine, p-phenylenediamine, diaminodiphenylmethane, diaminodiphenyl sulfone, 3,3′-diethyl-4,4′-diaminodiphenylmethane, maleic anhydride, succinic anhydride, tetrahydrophthalic anhydride, methyltetrahydrophthalic anhydride, methylnadic anhydride, hexahydrophthalic anhydride, methylhexahydrophthalic acid, methylcyclohexenetetracarboxylic anhydride, phthalic anhydride, trimellitic anhydride, benzophenonetetracarboxylic anhydride, dodecenylsuccinic anhydride, ethylene glycol bis(anhydrotrimellitate), phenol novolac resins, cresol novolac resins, poly(vinyl phenol), terpene-phenol resins, and polyamide resins.

Before a recording layer 13 is formed, a curing catalyst may be added to the polymeric matrix according to need. As the curing catalyst, a basic catalyst can be used. Examples thereof include tertiary amines, organic phosphine compounds, imidazole compounds, and derivatives thereof. Specifically, triethanolamine, piperidine, N,N′-dimethylpiperazine, 1,4-diazabicyclo(2,2,2)octane (triethylenediamine), pyridine, picoline, dimethylcyclohexylamine, dimethylhexylamine, benzyldimethylamine, 2-(dimethylaminomethyl)phenol, 2,4,6-tris(dimethylaminomethyl)phenol, DBU (1,8-diazabicyclo[5,4,0]undecan-7-ene) or the phenol salt thereof, trimethylphosphine, triethylphosphine, tributylphosphine, triphenylphosphine, tri(p-methylphenyl)phosphine, 2-methylimidazole, 2,4-dimethylimidazole, 2-ethyl-4-methylimidazole, 2-phenylimidazole, 2-phenyl-4-methylimidazole, 2-heptaimidazole, and the like can be used as a curing catalyst. Furthermore, a curing catalyst constituted of an aluminum tris(ethylacetylacetate)/triphenylsilanol mixed complex may be used to cure the polymeric matrix.

A latent catalyst also can be used. Examples thereof include boron trifluoride amine complexes, dicyandiamide, organic acid hydrazides, diaminomaleonitrile and derivatives thereof, melamine and derivatives thereof, and amine imides. It is also possible to add a compound having active hydrogen, such as, for example, phenol compounds or salicylic acid, to accelerate the curing of the three-dimensionally crosslinked polymeric matrix.

A polymerization inhibitor may have been dispersed in the recording layer according to need. A polymerization inhibitor is added for the purpose of inhibiting unexpected reactions from proceeding during storage. The polymerization inhibitor may have been freely diffused in the polymeric matrix or may have been fixed to the surface of the polymeric matrix through covalent bonds. Phenol derivatives, hindered amine compounds, and nitroxide compounds are preferred as the polymerization inhibitor. It is preferred to employ a polymerization inhibitor which does not inhibit polymeric matrix formation. However, this is not essential because the amount of the polymerization inhibitor is far smaller than the amount of the matrix and, hence, the polymerization inhibitor exerts little influence even if the inhibitor inhibits the reaction for polymeric matrix formation. However, in case where a polymerization inhibitor is deprived of the ability thereof by a reaction with a constituent ingredient for the polymeric matrix, it is necessary to select an adequate polymerization inhibitor from the choice of polymerization inhibitors shown above. In view of these, nitroxides, which have a polymerization-inhibiting site that is less apt to be affected by the polymeric matrix formation reaction, are especially suitable.

A nitroxide usually has a yellow to red color, and addition thereof to a recording layer colors the recording layer. It is, however, presumed that when the nitroxide recombines with a radical to become an alkoxyamine, the recording layer is decolored. Namely, the nitroxide traps radicals which have generated during storage and thereby changes into an alkoxylamine to become colorless. Also when the nitroxide traps radicals which have generated upon a pre-exposure treatment, the nitroxide changes into an alkoxylamine to become colorless. Namely, even if a colored recording layer is formed, this coloration does not exert an adverse influence on recording because the recording layer becomes transparent through dark reactions which occur during storage and through a pre-exposure treatment.

So long as a polymerization inhibitor has been incorporated in an amount of 0.0001 part by weight or more based on 100 parts by weight of the photopolymerization initiator, the effect thereof can be obtained. However, in case where a polymerization inhibitor has been incorporated in a higher concentration than the photopolymerization initiator, there is a possibility that the radicals which have generated from the photopolymerization initiator are wholly trapped by the polymerization inhibitor, making the recording impossible. It is therefore desirable to regulate the concentration of the polymerization inhibitor to a value which is up to 1 part by weight based on 100 parts by weight of the polymerization initiator. It is hence more preferred that the concentration of the polymerization inhibitor should be 0.0001-0.5 parts by weight based on 100 parts by weight of the photopolymerization initiator.

Specifically, the following compounds are more preferred as polymerization inhibitors: 2,2,6,6-tetramethylpiperidine N-oxide, 4-methacryloyl-2,2,6,6-tetramethylpiperidine N-oxide, 4-acryloyl-2,2,6,6-tetramethylpiperidine N-oxide, 4-methacrylamido-2,2,6,6-tetramethylpiperidine N-oxide, 4-acrylamido-2,2,6,6-tetramethylpiperidine N-oxide, 4-amino-2,2,6,6-tetramethylpiperidine N-oxide, 4-hydroxyl-2,2,6,6-tetramethylpiperidine N-oxide, 4-mercapto-2,2,6,6-tetramethylpiperidine N-oxide, 2,2,6,6-tetramethylpiperidine N-oxide 4-glycidyl ether, and the like.

A process for producing a hologram-recording medium 10 according to this embodiment is explained below.

For producing a hologram-recording medium 10 according to this embodiment, a recording-layer precursor solution is first prepared by mixing one or more polymerizable monomers including the polymerizable monomer represented by general formula (1), a photopolymerization initiator, and a polymeric matrix precursor.

A crosslinking agent, a sensitizer, and a plasticizer may be incorporated into the recording-layer precursor solution according to need. The recording-layer precursor solution obtained is used to form a resin layer on a transparent substrate, and a polymeric matrix is formed. Thus, a recording layer 13 is formed.

As the transparent substrate, for example, a glass substrate or a plastic substrate can be used. Prior to the application of the recording-layer precursor solution, the surface of the transparent substrate may be subjected to an adhesion-facilitating treatment selected from a corona discharge treatment, plasma treatment, ozone treatment, alkali treatment, and the like. For applying the solution, casting or spin coating can be employed. Alternatively, a recording layer 13 may be formed by disposing two transparent substrates through a resinous spacer and pouring the recording-layer precursor solution into the space therebetween.

In the case where an aliphatic primary amine was used as a curing agent, the reaction for polymeric matrix formation proceeds even at room temperature. According to the reactivity of the curing agent, the resin layer may be heated to a temperature of 30-150° C. The thickness of the recording layer 13 to be formed is preferably 20 μm to 2 mm, more preferably 50 μm to 2 mm. In case where the thickness of the recording layer is less than 20 μm, it is difficult to obtain a sufficient memory capacity. On the other hand, in case where the thickness of the recording layer 13 exceeds 2 mm, there is the possibility of decrease in sensitivity and diffraction efficiency.

Next, a method for recording on a hologram-recording medium 10 according to this embodiment and for read-out of the recorded information is explained.

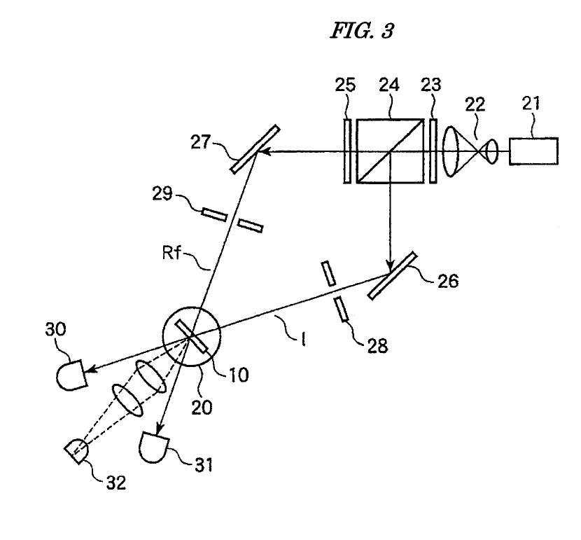

FIG. 3 shows a diagrammatic view of a hologram recording/read-out apparatus. The hologram recording/read-out apparatus shown in the figure is supposed to be a hologram recording/read-out apparatus which is operated by transmissive two-beam interferometry.

A light beam emitted from a light source device 21 passes through a beam expander 22 and an optical element for optical rotation 23 and is introduced into a polarized-beam splitter 24. As the light source device 21, it can be any light source which emits the desired light capable of interference in the recording layer 13 of a hologram-recording medium 10. From the standpoints of coherence, etc., linearly polarized laser is desirable. Examples of the laser include semiconductor lasers, He—Ne lasers, argon lasers, and YAG lasers.

The light emitted from the light source device 21 is expanded by the beam expander 22 to a beam diameter suitable for hologram recording. The optical element for optical rotation 23 optically rotates the light, the beam diameter of which has been enlarged by the beam expander 22, to thereby yield light which includes an S-polarized component and a P-polarized component. As the optical element for optical rotation 23, for example, a half-wave plate, quarter-wave plate, or the like can be used.

Of the light which has passed through the optical element for optical rotation 23, the S-polarized component is reflected by the polarized-beam splitter 24 and is then used as information light I. On the other hand, the P-polarized component passes through the polarized-beam splitter 24 and is used as reference light Rf.

Incidentally, the direction of optical rotation of the light which is incident on the polarized-beam splitter is regulated with the optical element for optical rotation 23 so that the information light I and the reference light Rf have the same intensity at the location of the recording layer 13 of the hologram-recording medium 10.

The information light I reflected by the polarized-beam splitter 24 is reflected by a mirror 26, subsequently passes through an electromagnetic shutter 28, and is irradiated upon the recording layer 13 of the hologram-recording medium 10 placed on a rotating stage 20.

On the other hand, the reference light Rf which has passed through the polarized-beam splitter 24 enters an optical element for optical rotation 25, by which the direction of polarization of the light is rotated by 90 degrees to give S-polarized light, which is reflected by a mirror 27. Thereafter, the reference light Rf passes through an electromagnetic shutter 29 and irradiated upon the recording layer 13 of the hologram-recording medium 10 placed on the rotating stage 20. In this recording layer 13, the reference light Rf intersects the information light I to generate interference fringes. Thus, a transmission hologram is formed in a refractive-index modulation region (not shown).

The recorded information is reproduced in the following manner. The electromagnetic shutter 28 is closed to thereby block the information light I and irradiate the reference light Rf only upon the transmission hologram (not shown) formed in the recording layer 13 of the hologram-recording medium 10. When the reference light Rf passes through the hologram-recording medium 10, part of the reference light Rf is diffracted by the transmission hologram. The resultant diffracted light is detected by a photodetector 30. Furthermore, a photodetector 31 has been disposed in order to detect the light which has passed through the medium.

For the purpose of illuminating and exposing the recording medium on which a hologram has been recorded, an ultraviolet light source device 32 and an optical system for ultraviolet irradiation can be disposed as shown in the figure. When the remaining unreacted polymerizable monomers are polymerized by means of these components, the higher stability of the recorded hologram can be obtained. As the ultraviolet light source device 32, any desired light source which emits light capable of polymerizing the unreacted polymerizable monomers can be used. When the efficiency of ultraviolet emission is taken into account, it is preferred to use, for example, a xenon lamp, mercury lamp, high-pressure mercury lamp, mercury-xenon lamp, gallium nitride-based light-emitting diode, gallium nitride-based semiconductor laser, excimer laser, the third harmonic of a Nd:YAG laser (355 nm), the fourth harmonic of a Nd:YAG laser (266 nm), or the like.

By thus using the hologram-recording medium according to this embodiment, a hologram-recording medium can be provided on which multiplexing recording is possible with high sensitivity.

Hologram-recording media according to this embodiment are evaluated below by reference to Examples and Comparative Examples.

### EXAMPLES 1 TO 26

Hologram-recording media according to the first embodiment were subjected to angular multiplexing recording, and were evaluated for M/# (M number), which indicates the dynamic range of recording.

For constituting a recording layer 13, a solution was made of 1,6-hexanediol diglycidyl ether as the main matrix ingredient and incorporating 0.5% by weight bis(cyclopenta-1,3-dienyl)bis(1-(2,4-difluoro)-3H-pyrrol-3-yl)titanium as a photopolymerization initiator and 3% by weight polymerizable monomer. A curing catalyst constituted of an aluminum tris(ethylacetylacetate)/triphenylsilanol mixed complex was dissolved in the solution to produce an even solution of a precursor for a hologram-recording medium.

The polymerizable monomer was represented by general formula (1) in which the 9-position and 6-position substituents of the carbazole moiety were fixed to a benzylvinyl group (BV) and a hydrogen atom, respectively, and the carbazole moiety had been substituted in the 3-position with any of iodine (I), bromine (Br), chlorine (Cl), methyl (Me), ethyl (Et), isopropyl (iPr), tert-butyl (tBu), phenyl (Ph), naphthyl (Np), hydroxyl (OH), methoxy (O-Me), ethoxy (O-Et), isopropoxy (O-iPr), tert-butoxy (O-tBu), phenoxy (O-Ph), naphthoxy (O—Np), acetyl (C(═O)-Me), carboxyl (C(═O)—OH), acetoxy (C(═O)—OMe), thiophenyl (S-Ph), thionaphthyl (S—Np), thiomethyl (S-Me), thioethyl (S-Et), thioisopropyl (S-iPr), thio-tert-butyl (S-tBu), and thiol (SH).

Two glass substrates were disposed through a spacer constituted of a sheet made of polytetrafluoroethylene (PTFE) to thereby form a space therebetween. The solution of a precursor for a hologram-recording medium was poured into the space. This structure was shielded from light, and the solution was cured by heating for 24 hours. Thus, a hologram-recording medium sample having a recording layer with a thickness of 200 μm was produced. Through the steps described above, hologram-recording media 10 having the configuration shown in FIG. 1 were obtained.

Each sample produced was placed on the rotating stage 20 of the hologram recording/read-out apparatus shown in FIG. 3, and holograms were recorded thereon. As the light source device 21, a semiconductor laser having a wavelength of 405 nm was used. Angular multiplexing recording was conducted with respect to a plurality of positions on the test piece to evaluate the hologram-recording performance in terms of M/# (M number), which indicates the dynamic range of recording. In this operation, a pre-exposure treatment was conducted in an even exposure amount just before each recording step. As shown by the following equation (1), M/# is defined using ηᵢ. Symbol ηᵢ is the efficiency of the diffraction obtained from the i-th hologram when n-page holograms were recorded on the same region within the recording layer of the hologram-recording medium and read out by angular multiplexing recording/read-out until the recording became impossible. The angular multiplexing recording/read-out is conducted by irradiating given light upon the transmission type hologram-recording medium 10 while operating the rotating stage 20.

$$M \text{/} \# = {\sum }_{i = 1}^{n} \sqrt{η} i \qquad Equation ( 1 )$$

Incidentally, the efficiency of diffraction η was defined using the light intensity I measured when the reference light Rf only was irradiated onto the transmission type hologram-recording medium 10 and using the intensity I_{d} of the light detected by the photodetector 30. Namely, the external diffraction efficiency represented by η=I_{d}/I was used.

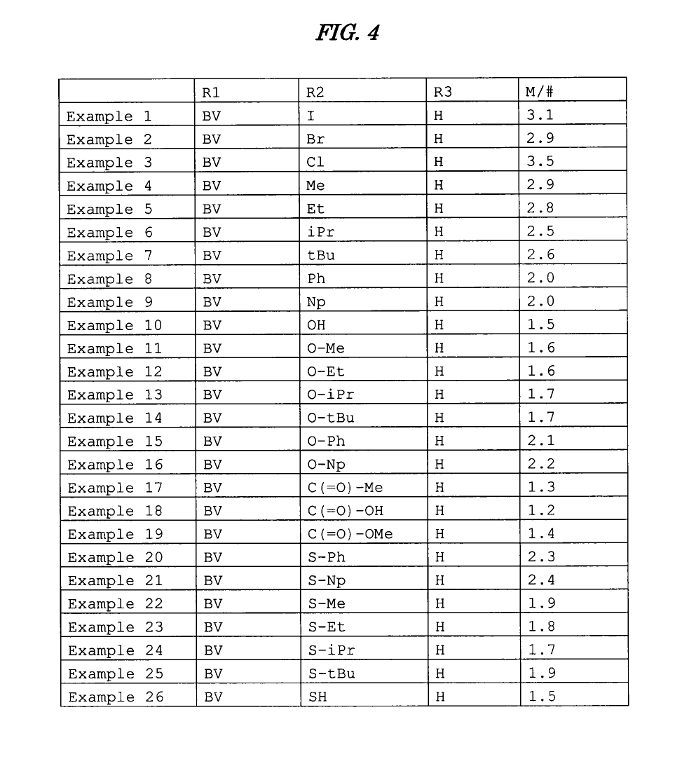

The results of the evaluation for M number of the hologram-recording media according to the Examples are shown in FIG. 4, in which W indicates the 9-position of the carbazole moiety, X indicates the 6-position of the carbazole moiety, and Y indicates the 3-position of the carbazole moiety.

As demonstrated by Examples 1 to 26, the substitution in the 9-position of the carbazole moiety with a benzylvinyl group and the use of different substituents in the 3-position and 6-position of the carbazole moiety resulted in an M number of 1 or larger in all Examples.

### EXAMPLES 27 TO 52

Hologram-recording media 10 according to the first embodiment were evaluated for M number in the same manner as in Examples 1 to 26.

Examples 27 to 52 differed from Examples 1 to 26 in that the carbazole moiety represented by general formula (1) had been substituted in the 9-position with a styryl group (S). Except this, Examples 27 to 52 were the same as Examples 1 to 26. An explanation hence is omitted.

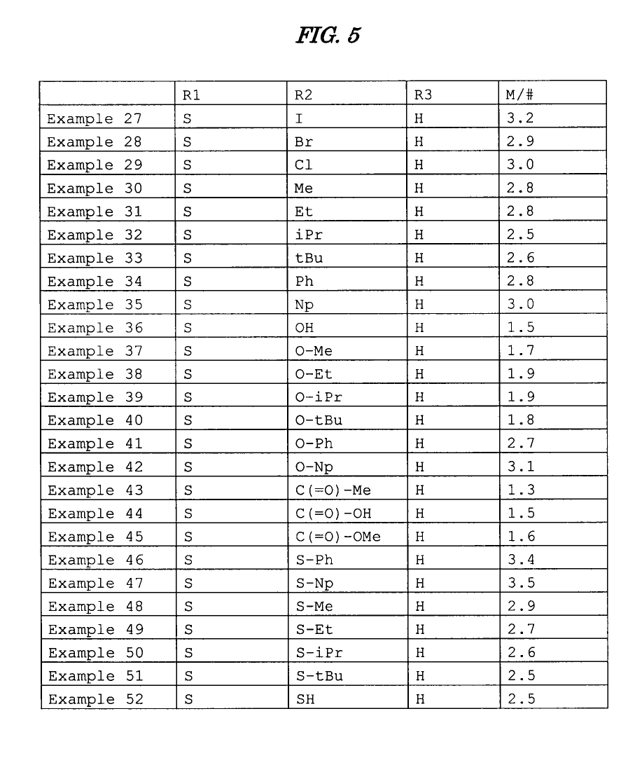

In FIG. 5 are shown the results of the evaluation for M number of the hologram-recording media according to the Examples. W indicates the 9-position of the carbazole moiety, X indicates the 6-position of the carbazole moiety, and Y indicates the 3-position of the carbazole moiety.

As demonstrated by Examples 27 to 52, the substitution in the 9-position of the carbazole moiety with a styryl group and the use of different substituents in the 3-position and 6-position of the carbazole moiety resulted in an M number of 1 or larger in all Examples.

### EXAMPLES 53 TO 78

Hologram-recording media 10 according to the first embodiment were evaluated for M number in the same manner as in Examples 1 to 26.

Examples 53 to 78 differed from Examples 1 to 26 in that the carbazole moiety represented by general formula (1) had been substituted in the 9-position with an acryloyl group (A). Except this, Examples 53 to 78 were the same as Examples 1 to 26. An explanation hence is omitted.

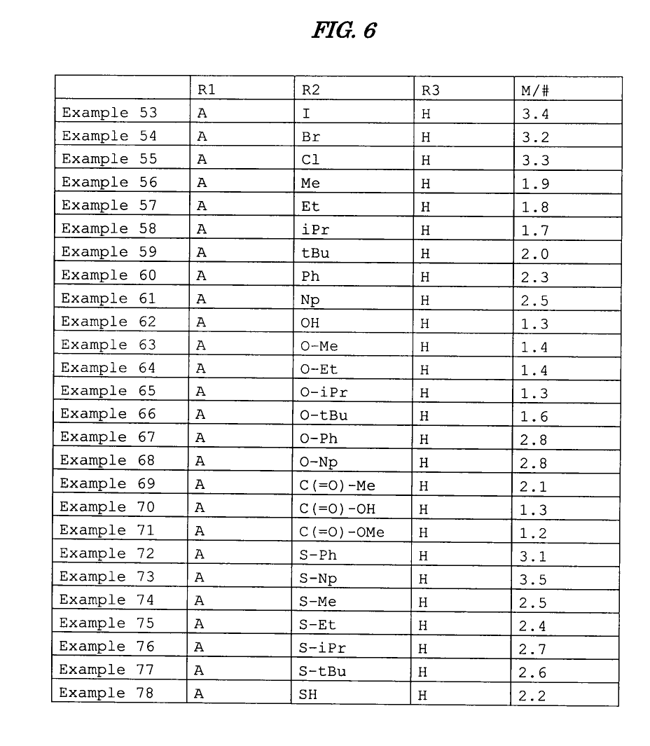

In FIG. 6 are shown the results of the evaluation for M number of the hologram-recording media according to the Examples. W indicates the 9-position of the carbazole moiety, X indicates the 6-position of the carbazole moiety, and Y indicates the 3-position of the carbazole moiety.

As demonstrated by Examples 53 to 78, the substitution in the 9-position of the carbazole moiety with an acryloyl group and the use of different substituents in the 3-position and 6-position of the carbazole moiety resulted in an M number of 1 or larger in all Examples.

### EXAMPLES 79 TO 104

Hologram-recording media 10 according to the first embodiment were evaluated for M number in the same manner as in Examples 1 to 26.

Examples 79 to 104 differed from Examples 1 to 26 in that the carbazole moiety represented by general formula (1) had been substituted in the 9-position with a methacryloyl group (MA). Except this, Examples 79 to 104 were the same as Examples 1 to 26. An explanation hence is omitted.

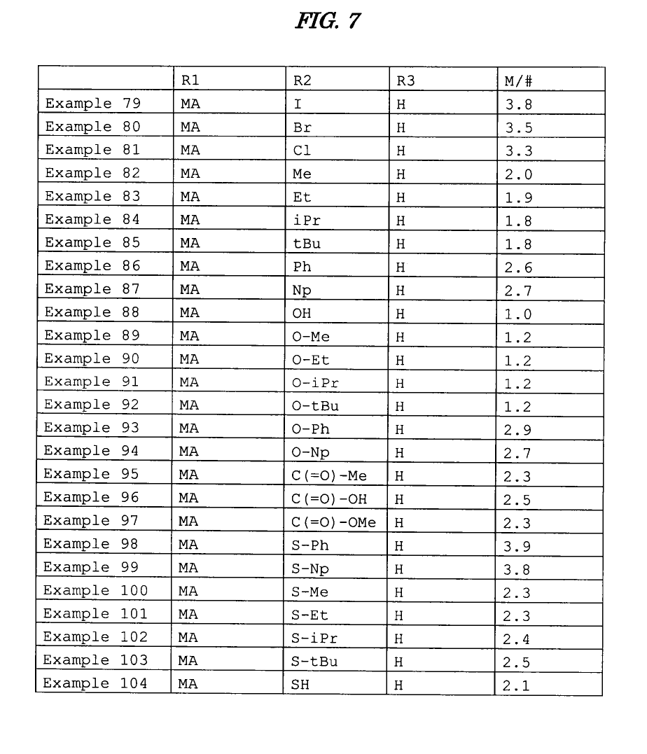

In FIG. 7 are shown the results of the evaluation for M number of the hologram-recording media according to the Examples. W indicates the 9-position of the carbazole moiety, X indicates the 6-position of the carbazole moiety, and Y indicates the 3-position of the carbazole moiety.

As demonstrated by Examples 79 to 104, the substitution in the 9-position of the carbazole moiety with a methacryloyl group and the use of different substituents in the 3-position and 6-position of the carbazole moiety resulted in an M number of 1 or larger in all Examples.

### COMPARATIVE EXAMPLES 1 TO 4

Hologram-recording media 10 were evaluated for M number in the same manner as in Examples 1 to 26.

Comparative Examples 1 to 4 differed from Examples 1 to 26 in that the carbazole moiety represented by general formula (1) had been substituted in the 9-position with any of a benzylvinyl group (BV), a styryl group (S), an acryloyl group (A), and a methacryloyl group (MA) and that the carbazole moiety had the same substituent in the 3-position and in the 6-position. Except this, Comparative Examples 1 to 4 were the same as Examples 1 to 26. An explanation hence is omitted. In the experiments made here, iodine (I) was employed, as an example, as each of the 3-position and 6-position substituents of the carbazole moiety.

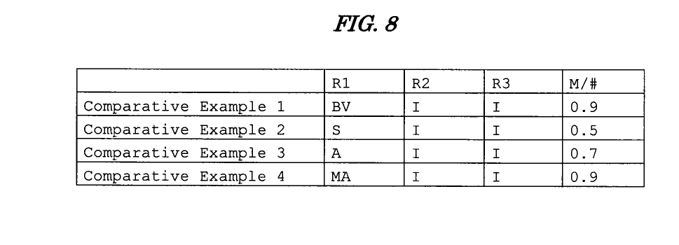

In FIG. 8 are shown the results of the evaluation for M number of the hologram-recording media according to the Comparative Examples. W indicates the 9-position of the carbazole moiety, X indicates the 6-position of the carbazole moiety, and Y indicates the 3-position of the carbazole moiety.

As Comparative Examples 1 to 4 show, the substitution in the 9-position of the carbazole moiety with any of a benzylvinyl group (BV), a styryl group (S), an acryloyl group (A), and a methacryloyl group (MA) and the use of same substituent in the 3-position and 6-position of the carbazole moiety resulted, in all Comparative Examples, in an M number which was smaller than in Examples 1 to 104 and was smaller than 1.

It was found from those results that compared to Comparative Examples 1 to 4, in which the same substituent had been used in the 3-position and 6-position of the carbazole moiety, the hologram-recording medium samples of Examples 1 to 104, in which the carbazole moiety had had different substituents in the 3-position and the 6-position, showed a higher value of M/# even when the irradiation energy amount was the same.

The reasons for this are as follows. When carbazole molecules having the same substituent in the 3-position and the 6-position are polymerized, electrostatic repulsion occurs between the carbazole molecules, making it difficult to construct a relationship in which the surface irregularities of carbazole rings are mated with the surface irregularities of other carbazole moiety. On the other hand, when the 3-position substituent differs from the 6-position substituent, such electrostatic repulsion can be diminished as compared with the case where the 3-position substituent is the same as the 6-position substituent, thereby making it easy to construct a relationship in which the surface irregularities of carbazole rings are mated with the surface irregularities of other carbazole rings.

As demonstrated above, by bonding different substituents in the 3-position and the 6-position, the following effect is obtained. When layers of this polymerizable monomer are stacked so as to be oriented reversely, a relationship can be constructed in which the surface irregularities of the carbazole rings in one layer are mated with the surface irregularities of the carbazole rings in the other layer. As a result, layers of the polymerizable monomer can be stacked densely as compound with the case where the same substituent has been bonded in the 3-position and the 6-position, and this region can be made to have a further increased actual refractive index after recording, i.e., after polymerization reaction. As a result, a high degree of refractive-index modulation can be brought about through a smaller number of polymerization reaction operations.

It can hence be seen that the hologram-recording media according to this embodiment are capable of multiplexing recording with higher sensitivity than conventional hologram-recording media.

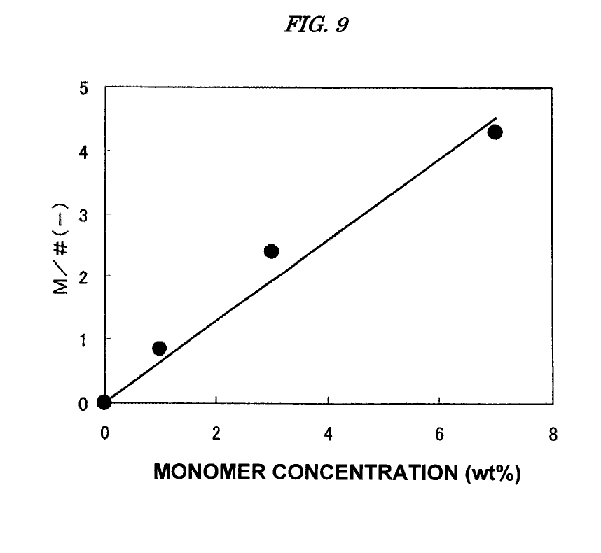

In the series of evaluation, the polymerizable-monomer concentration was set at 3% by weight in all evaluation. However, it was found from experiments made hitherto that monomer concentration is proportional to M/#, as shown in FIG. 9. It is hence thought that even if concentrations different from that value were used for the evaluation, the hologram-recording media would show M/# values respectively corresponding to the concentrations.

Although the embodiments according to the present invention have been described above, the present invention is not limited to the above-mentioned embodiments but can be variously modified. Constituent components disclosed in the aforementioned embodiments may be combined suitably to form various modifications. For example, some of all constituent components disclosed in the embodiments may be removed or may be appropriately combined.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as described by the appended claims and their equivalents.

What is claimed is:

1. A hologram-recording medium comprising:
a recording layer comprising a polymerizable monomer containing a structural framework represented by the following general formula (1). 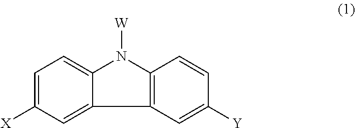
In general formula (1), X and Y are not the same, and X and Y are selected from hydrogen, iodine, bromine, and chlorine atoms and from methyl, ethyl, isopropyl, tert-butyl, phenyl, naphthyl, hydroxyl, methoxy, ethoxy, isopropoxy, tert-butoxy, phenoxy, naphthoxy, acetyl, carboxyl, acetoxy, thiophenyl, thionaphthyl, thiomethyl, thioethyl, thioisopropyl, thio-tert-butyl, and thiol groups; and W is selected from the group consisting of benzylvinyl, styryl, acryloyl, and methacryloyl groups.

2. The hologram-recording medium according to claim 1, further comprising:
a pair of transparent substrates, between which the recording layer is held.

3. The hologram-recording medium according to claim 2, further comprising:
a reflecting layer disposed between either one of the transparent substrates and the recording layer and
a gap layer formed between the reflecting layer and the recording layer.

4. The hologram-recording medium according to claim 3, wherein the gap layer is made of transparent material.
